SEQUENCE LISTING (15 sequences)

SEQ ID NO: 1 - DNA, 732 bp, Homo sapiens
  CDS (74)..(442): minK-related peptide 1, Val substitued for Ala at amino acid 116
caaatccagaaaagatccgttttcctaaccttgttcgcctattttattatttaaattgcagcaggagggaagcatgtctactttatccaatttcacacagacgctggaagacgtcttccgaaggatttttattacttatatggacaattggcgccagaacacaacagctgagcaagaggccctccaagccaaagttgatgctgagaacttctactatgtcatcctgtacctcatggtgatgattggaatgttctctttcatcatcgtggccatcctggtgagcactgtgaaatccaagagacgggaacactccaatgacccctaccaccagtacattgtagaggactggcaggaaaagtacaagagccaaatcttgaatctagaagaatcgaaggccaccatccatgagaacattggtgtggctgggttcaaaatgtccccctgataagggagaaaggcaccaagctaacatctgacgtccagacatgaagagatgccagtgccacgaggcaaatccaaattgtctttgcttagaagaaagtgagttccttgctctttgttgagaattttcatggagattatgtggttggccaataaagatagatgacatttcaatctcagtgatttatgcttgcttgttggagcaatattttgtgctgaagacctcttttactttccgggcaagtgaatgtcattttaatcaatatcaatgatgaaaataaagccaaattt

SEQ ID NO: 2 - protein, 123 aa, Homo sapiens
MSTLSNFTQTLEDVFRRIFITYMDNWRQNTTAEQEALQAKVDAENFYYVILYLMVMIGMFSFIIVAILVSTVKSKRREHSNDPYHQYIVEDWQEKYKSQILNLEESKATIHENIGVAGFKMSP

SEQ ID NO: 3 - DNA, 732 bp, Homo sapiens
  CDS (74)..(442): minK-related peptide 1, wild type
caaatccagaaaagatccgttttcctaaccttgttcgcctattttattatttaaattgcagcaggagggaagcatgtctactttatccaatttcacacagacgctggaagacgtcttccgaaggatttttattacttatatggacaattggcgccagaacacaacagctgagcaagaggccctccaagccaaagttgatgctgagaacttctactatgtcatcctgtacctcatggtgatgattggaatgttctctttcatcatcgtggccatcctggtgagcactgtgaaatccaagagacgggaacactccaatgacccctaccaccagtacattgtagaggactggcaggaaaagtacaagagccaaatcttgaatctagaagaatcgaaggccaccatccatgagaacattggtgcggctgggttcaaaatgtccccctgataagggagaaaggcaccaagctaacatctgacgtccagacatgaagagatgccagtgccacgaggcaaatccaaattgtctttgcttagaagaaagtgagttccttgctctttgttgagaattttcatggagattatgtggttggccaataaagatagatgacatttcaatctcagtgatttatgcttgcttgttggagcaatattttgtgctgaagacctcttttactttccgggcaagtgaatgtcattttaatcaatatcaatgatgaaaataaagccaaattt

SEQ ID NO: 4 - protein, 123 aa, Homo sapiens
MSTLSNFTQTLEDVFRRIFITYMDNWRQNTTAEQEALQAKVDAENFYYVILYLMVMIGMFSFIIVAILVSTVKSKRREHSNDPYHQYIVEDWQEKYKSQILNLEESKATIHENIGAAGFKMSP

SEQ ID NO: 5 - DNA, 732 bp, Homo sapiens
  CDS (74)..(442): minK-related peptide 1, Thr substitued for Met at amino acid 54
caaatccagaaaagatccgttttcctaaccttgttcgcctattttattatttaaattgcagcaggagggaagcatgtctactttatccaatttcacacagacgctggaagacgtcttccgaaggatttttattacttatatggacaattggcgccagaacacaacagctgagcaagaggccctccaagccaaagttgatgctgagaacttctactatgtcatcctgtacctcacggtgatgattggaatgttctctttcatcatcgtggccatcctggtgagcactgtgaaatccaagagacgggaacactccaatgacccctaccaccagtacattgtagaggactggcaggaaaagtacaagagccaaatcttgaatctagaagaatcgaaggccaccatccatgagaacattggtgcggctgggttcaaaatgtccccctgataagggagaaaggcaccaagctaacatctgacgtccagacatgaagagatgccagtgccacgaggcaaatccaaattgtctttgcttagaagaaagtgagttccttgctctttgttgagaattttcatggagattatgtggttggccaataaagatagatgacatttcaatctcagtgatttatgcttgcttgttggagcaatattttgtgctgaagacctcttttactttccgggcaagtgaatgtcattttaatcaatatcaatgatgaaaataaagccaaattt

SEQ ID NO: 6 - protein, 123 aa, Homo sapiens
MSTLSNFTQTLEDVFRRIFITYMDNWRQNTTAEQEALQAKVDAENFYYVILYLTVMIGMFSFIIVAILVSTVKSKRREHSNDPYHQYIVEDWQEKYKSQILNLEESKATIHENIGAAGFKMSP

SEQ ID NO: 7 - DNA, 732 bp, Homo sapiens
  CDS (74)..(442): minK-related peptide 1, Thr substituted for Ile at amino acid 57
caaatccagaaaagatccgttttcctaaccttgttcgcctattttattatttaaattgcagcaggagggaagcatgtctactttatccaatttcacacagacgctggaagacgtcttccgaaggatttttattacttatatggacaattggcgccagaacacaacagctgagcaagaggccctccaagccaaagttgatgctgagaacttctactatgtcatcctgtacctcatggtgatgactggaatgttctctttcatcatcgtggccatcctggtgagcactgtgaaatccaagagacgggaacactccaatgacccctaccaccagtacattgtagaggactggcaggaaaagtacaagagccaaatcttgaatctagaagaatcgaaggccaccatccatgagaacattggtgcggctgggttcaaaatgtccccctgataagggagaaaggcaccaagctaacatctgacgtccagacatgaagagatgccagtgccacgaggcaaatccaaattgtctttgcttagaagaaagtgagttccttgctctttgttgagaattttcatggagattatgtggttggccaataaagatagatgacatttcaatctcagtgatttatgcttgcttgttggagcaatattttgtgctgaagacctcttttactttccgggcaagtgaatgtcattttaatcaatatcaatgatgaaaataaagccaaattt

SEQ ID NO: 8 - protein, 123 aa, Homo sapiens [sequence omitted]

SEQ ID NO: 9 - DNA, 732 bp, Homo sapiens
  CDS (74)..(442): minK-related peptide 1, Ala substitued for Thr at amino acid 8
caaatccagaaaagatccgttttcctaaccttgttcgcctattttattatttaaattgcagcaggagggaagcatgtctactttatccaatttcgcacagacgctggaagacgtcttccgaaggatttttattacttatatggacaattggcgccagaacacaacagctgagcaagaggccctccaagccaaagttgatgctgagaacttctactatgtcatcctgtacctcatggtgatgattggaatgttctctttcatcatcgtggccatcctggtgagcactgtgaaatccaagagacgggaacactccaatgacccctaccaccagtacattgtagaggactggcaggaaaagtacaagagccaaatcttgaatctagaagaatcgaaggccaccatccatgagaacattggtgcggctgggttcaaaatgtccccctgataagggagaaaggcaccaagctaacatctgacgtccagacatgaagagatgccagtgccacgaggcaaatccaaattgtctttgcttagaagaaagtgagttccttgctctttgttgagaattttcatggagattatgtggttggccaataaagatagatgacatttcaatctcagtgatttatgcttgcttgttggagcaatattttgtgctgaagacctcttttactttccgggcaagtgaatgtcattttaatcaatatcaatgatgaaaataaagccaaattt

SEQ ID NO: 10 - protein, 123 aa, Homo sapiens
MSTLSNFAQTLEDVFRRIFITYMDNWRQNTTAEQEALQAKVDAENFYYVILYLMVMIGMFSFIIVAILVSTVKSKRREHSNDPYHQYIVEDWQEKYKSQILNLEESKATIHENIGAAGFKMSP

SEQ ID NO: 11 - DNA, 21 bp, Artificial Sequence
  Description of Artificial Sequence  PCR amplification primer
tgttcgcctattttattattt

SEQ ID NO: 12 - DNA, 20 bp, Artificial Sequence
  Description of Artificial Sequence  PCR amplification primer
aaagagaacattccaatcat

SEQ ID NO: 13 - DNA, 21 bp, Artificial Sequence
  Description of Artificial Sequence  PCR amplification primer
tactatgtcatcctgtacctc

SEQ ID NO: 14 - DNA, 19 bp, Artificial Sequence
  Description of Artificial Sequence  PCR amplification primer
gttagcttggtgcctttct

SEQ ID NO: 15 - DNA, 20 bp, Artificial Sequence
  Description of Artificial Sequence  PCR amplification primer
aacattggtgtggctgggtt

(19) United States
(12) Patent Application Publication
(10) Pub. No.: US 2004/0086886 A1
(43) Pub. Date: May 6, 2004

(54) Polymorphisms associated with cardiac arrythmia

(76) Inventor: Steven A.N. Goldstein, Guilford, CT (US)

Correspondence Address: MORGAN LEWIS & BOCKIUS LLP, 1111 PENNSYLVANIA AVENUE NW, WASHINGTON, DC, 20004, US

(21) Appl. No.: 10/380,111

(22) PCT Filed: Sep. 11, 2001
(86) PCT No.: PCT/US01/28332

(51) Int. Cl. C12Q 1/68; C07H 21/04; C12P 21/02; C12N 1/21; C12N 5/06; C07K 14/705

(52) U.S. Cl. 435/6; 435/69.1; 435/320.1; 435/358; 435/252.3; 530/350; 536/23.5

(57) ABSTRACT

The invention relates generally to the discovery of specific nucleotide polymorphisms in the KCNE2 gene and the association of these polymorphisms with antibiotic-induced LQTS. Related composition screening systems and diagnostic and prognostic assays are provided.

### FIELD OF THE INVENTION

The invention relates generally to genes encoding the Min-K related (MiRP) ion channel protein subunits and polymorphisms in these genes that are associated with ion channel disorders, including antibiotic-induced long QT syndrome (LQTS).

### BACKGROUND OF THE INVENTION

Cardiac arythmias are a common cause of morbidity and mortality, accounting for approximately 11% of all natural deaths (Kannel et al. (1987) Am. Heart J. 113, 799-804; Willich et al. (1987) Am. J. Cardiol. 60, 801-806). In general, presymptomatic diagnosis and treatment of individuals with life-threatening ventricular tachyarrhythmias is poor, and in some cases medical management actually increases the risk of arrhythmia and death (Cardiac Arrhythmia Suppression Trial II Investigators (1992) N. Engl. J. Med. 327, 227-233). These factors make early detection of individuals at risk for cardiac arhythmias and arrhythmia prevention high priorities.

Long QT Syndrome

Long QT syndrome (LQTS) is a type of cardiac arrhythmia that causes abrupt loss of consciousness, syncope, seizures and sudden death from tachyarrhythmias, specifically torsades de pointes and ventricular fibrillation (Ward (1964) J. Ir. Med. Assoc. 54, 103-106; Schwartz et al. (1975) Am. Heart J. 109, 378-390; Moss et al. (1991) Circulation 84, 1136-1144. Torsade depointes (TdP) may degenerate into ventricular fibrillation, a particularly lethal arrhythmia. Although LQT is not a common diagnosis, ventricular arrhythmias are very common; more than 300,000 United States citizens die suddenly every year (Kannel et al. (1987) Am. Heart J. 113, 799-804; Willich et al. (1987) Am. J. Cardiol. 60, 801-806) and, in many cases, the underlying mechanism may be aberant cardiac repolarization. Both inherited and acquired forms of LQTS have been identified. Acquired LQT and secondary arrhythmias can result from cardiac ischemia, bradycardia and metabolic abnormalities such as low serum potassium or calcium concentration (Zipes (1987) Am. J. Cardiol. 59, 26E-31E). LQTS can also result from treatment with certain medications, including antibiotics, antihistamines, general anesthetics, and, most commonly, antiarrhythmic medications (Zipes (1987) Am. J. Cardiol. 59, 26E-31E).

Molecular Basis for LQTS

The molecular basis for LQTS is known: delayed repolarization of the myocardium prolongs the cardiac action potential increasing the QT interval measured on the surface electrocardiogram (ECG). Mutations in five ion channel genes cause the majority of cases of inherited LQTS. LQTS mutations in SCN5A increase the conductance of the sodium channel that depolarizes the myocardium to initiate the cardiac action potential (Bennett et al. (1995) Nature 376, 683-685). LQTS mutations in KCNE1 or KvLQT1 (encoding subunits of I_{Ks} channels) and KCNE2 or HERG (encoding subunits of I_{Kr} channels) diminish potassium fluxes that repolarize the heart to end each beat (Curran et al. (1995) Cell 80, 795-803; Wang et al. (1996) Nat. Genet. 12, 17-23; Splawski et al. (1997) Nat. Genet. 17, 338-340; Abbott et al. (1999) Cell 97, 175-187). In addition, specific sporadic mutations have been identified in patients with drug-induced TdP (Abbott et al. (1999) Cell 97, 175-187; Donger et al. (1997) Circulation 96, 2778-2781). Thus, it appears that patients with “acquired” LQTS have a genetic predisposition to arrhythmia due to mutation in the MiRP1 subunit (encoded by KCNE2) of their I_{Kr} potassium channels (Abbott et al. (1999) Cell 97, 175-187). Previously, clarithromycin-induced TdP was associated with a sporadic missense mutation in KCNE2; channels formed with the altered subunit (Q9E-MiRP1) were abnormal at baseline because they activated less readily (and, so, passed less potassium); moreover, Q9E-MiRP1 channels were three-fold more sensitive to drug inhibition than wild type (Abbott et al. (1999) Cell 97, 175-187). In that case, a rare mutation increased the risk of drug-induced TdP both by decreasing repolarization reserve and increasing sensitivity to an agent that is usually well-tolerated (Abbott et al. (1999) Cell 97, 175-187).

Although the scientific and medical literature has identified several genes and their corresponding proteins in both inherited and acquired forms of LQTS, identification of the specific genetic defects that predispose individuals to drug-induced, and specifically, antibiotic-induced LQTS, has been lacking. Currently, most presymptomatic diagnosis of LQTS is based on the prolongation of the QT interval on electrocardiograms. Most patients with LQTS, however, are young, otherwise healthy individuals, who generally do not receive routine electrocardiograms. This diagnostic paradox illustrates the necessity of specific and reliable diagnostic tests to identify persons at risk for such disorders. Similarly, what has been absent from the therapeutic perspective are compositions that modulate the activity of aberrant ion channels that lead to life threatening arrythmias. It is to such diagnostic kits, prognostic testing and therapeutic compositions and to related compound screening systems that the discoveries of the present inventors are directed.

### SUMMARY OF THE INVENTION

The present invention is based on the discovery of specific nucleotide polymorphisms in the KCNE2 gene and the association of these polymorphisms with antibiotic-induced LQTS.

The invention thus encompasses an isolated polynucleotide sequence selected from the group consisting of (a) a nucleotide sequence that encodes an MiRP1 polypeptide that contains a mutation that causes long QT syndrome (LQTS), or biologically active fragments thereof that retain the mutation; (b) a nucleotide sequence that encodes an MiRP1 polypeptide that contains a mutation at the amino acid corresponding to position 116 of the MiRP1 polypeptide described by SEQ ID NO: 4, or biologically active fragments thereof that retain the mutation; (c) a nucleotide sequence that encodes an MiRP1 polypeptide that wherein the amino acid corresponding to position 116 of the MiRP1 polypeptide described by SEQ ID NO: 4 is valine or, or biologically active fragments thereof that retain the mutation; (d) a nucleotide sequence that encodes an MiRP1 polypeptide described by SEQ ID NO: 2, or biologically active fragments thereof that retain the mutation; and (e) a nucleotide sequence that is described by SEQ ID NO: 1, or fragments thereof that retain the mutation.

The invention further encompasses a vector comprising the aforementioned polynucleotide, a cell transfected with the aforementioned polynucleotide or vector. In one embodiment the cell is prokaryotic while in another embodiment it is eukcaryotic. In a preferred embodiment the cell is a Chinese Hamster Ovary (CHO) cell. The invention also includes a method of producing a polypeptide comprising the steps of culturing the cell transfected with any of the polynucleotides of the invention and isolating the polypeptide.

The invention also encompasses an isolated polypeptide selected from the group consisting of (a) a polypeptide comprising the amino acid sequence described by SEQ ID NO: 4 wherein the amino acid at position 116 is not alanine; (b) a polypeptide comprising the amino acid sequence described by SEQ ID NO: 4 wherein the amino acid at position 116 is valine; and (c) a polypeptide encoded by any of the polynucleotides of the invention.

The invention includes nucleic acid probes that specifically hybridize to the polynucleotide described by SEQ ID NO: 1, SEQ ID NO: 5, SEQ ID NO: 7 or SEQ ID NO: 9 under stringent hybridization conditions wherein said hybridization conditions prevent said nucleic acid probe from hybridizing to a polynucleotide comprising SEQ ID NO: 1, SEQ ID NO: 3. In a preferred embodiment, this nucleic acid probe comprises at least 10 contiguous nucleotides of SEQ ID NO: 1, SEQ ID NO: 5, SEQ ID NO: 7 or SEQ ID NO: 9. in a preferred embodiment, the nucleic acid probe consists of between 10-100 nucleotides. In another embodiment, the invention includes a kit comprising any one of the nucleic acid probes of the invention.

The invention also includes a method for diagnosing a polymorphism that causes drug-induced LQTS, comprising the step of hybridizing any one of the aforementioned nucleic acid probes to an isolated sample of a patient's DNA or RNA, under stringent hybridization conditions that allow hybridization of the probe to a nucleic acid comprising said polymorphism but which prevent hybridization of said probe to the polynucleotide described by SEQ ID NO: 3, wherein the presence of a hybridization signal indicates the presence of said polymorphism. In some embodiments, the patient's DNA or RNA has been amplified and said amplified DNA or RNA is hybridized. In a preferred embodiment, the hybridization is performed in situ.

The invention further includes a method for diagnosing a polymorphism that causes drug-induced LQTS, comprising the DNA sequencing of a patient's KCNE2 gene, wherein a mutation at a nucleotide position encoding an amino acid residue corresponding to positions 8, 54, 57, or 116 of the MiRP1 polypeptide (SEQ ID NO: 4), indicates the presence of said polymorphism.

The invention encompasses an antibody that binds to a mutant MiRP1 polypeptide but not to the polypeptide described by SEQ ID NO: 4. In a preferred embodiment, the antibody binds to a polypeptide comprising SEQ ID NO: 2, SEQ ID NO: 6, SEQ ID NO: 8 and/or SEQ ID NO: 10. In a more preferred embodiment, the antibody is monoclonal. The antibodies of the invention may be used to detect the presence of a positive reaction, such detection indicating a polymorphism.

The invention further encompasses a nonhuman, transgenic animal comprising any one of the polynucleotides of the invention.

The invention includes a method of detecting compounds that are useful in treating or preventing LQTS, said method comprising (a) placing cells expressing wild-type HERG and wild-type KCNE2 into a bathing solution to measure current; (b) measuring an induced K+ current in the cells of step (a); (c) placing cells expressing wild-type HERG and mutant KCNE2 into a bathing solution to measure current; (d) measuring the induced K+ current in cells of step (c); (e) adding a candidate compound to the bathing solution of step (c); (f) measuring an induced K+ current in the cells of step (e); and (g) determining whether said candidate compound produces an induced K+ current more or less similar to the induced K+ current seen in cells expressing wild-type HERG and wild-type KCNE2 as compared to the current seen in cells expressing wild-type HERG and mutant KCNE2 in the absence of said candidate compound, wherein the candidate compound that produces a current more similar to the current seen in cells expressing wild-type HERG and wild-type KCNE2 is useful in treating or preventing LQTS. In some embodiments, the mutant KCNE2 is selected from the group consisting of (a) a polynucleotide that encodes a mutant MiRP1 polypeptide described by SEQ ID NO: 2; (b) a polynucleotide that encodes a mutant MiRP1 polypeptide described by SEQ ID NO: 6; (c) a polynucleotide that encodes a mutant MiRP1 polypeptide described by SEQ ID NO: 8; (d) a polynucleotide that encodes a mutant MiRP1 polypeptide described by SEQ ID NO: 10; (e) a polynucleotide described by SEQ ID NO: 1; (f) a polynucleotide described by SEQ ID NO: 5; (g) a polynucleotide described by SEQ ID NO: 7; and (h) a polynucleotide described by SEQ ID NO: 9.

### BRIEF DESCRIPTION OF THE DRAWINGS

FIG. 1: Schematic of the predicted primary sequence and membrane topology of human MiRP1 (SEQ ID NO: 4).

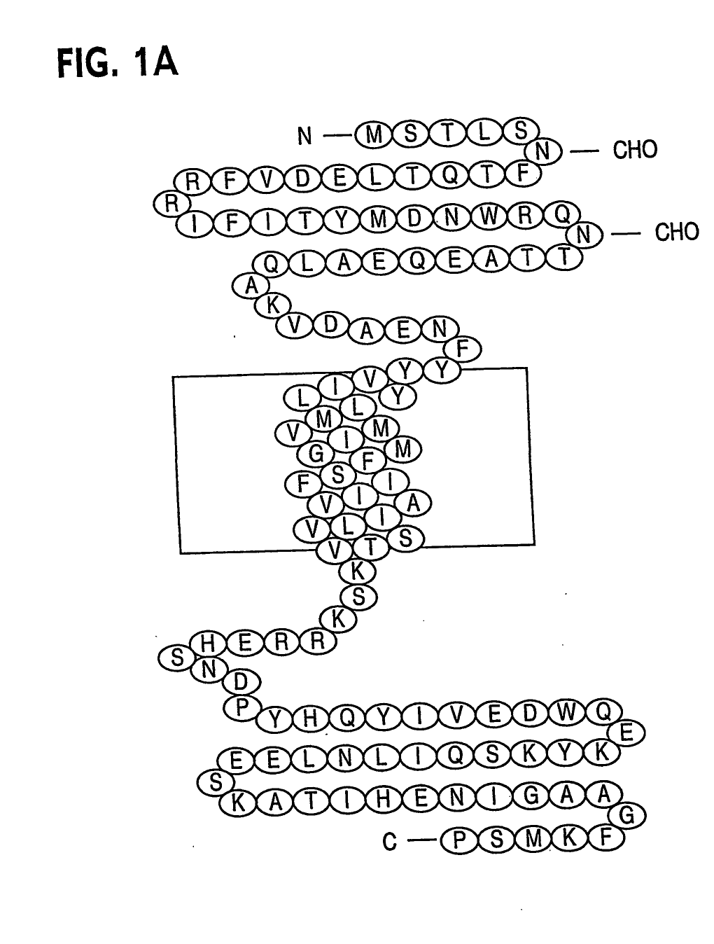

FIG. 2: Current density assessed by normalizing steady-state tail currents at −40 mV to the cell capacitance for CHO cells expressing the indicated MiRP1 variants and HERG and studied by the standard protocol (materials and Methods). Each bar represents the mean±SEM for a group of 34-40 cells; the mean decrease in current density compared to wild type MiRP1 was 15, 39, 34 and 47% for T8A-MiRP1, M54T-MiRP1, I57T-and A116V-MiRP1, respectively, with p<10⁻⁴ for each variant compared to wild type (unpaired t-test).

FIG. 3: I_{Kr} channels formed with A116V-MiRP1 show wild type blockade by quinidine. CHO cells expressing wild type MiRP1 or A116V-MiRP1 and HERG were studied by whole-cell clamp. (A) Whole-cell currents recorded from channels formed with wild type MiRP1 (WT) and A116V-MiRP1 (A116V) in the absence (left column) or presence of 0.005 mg/ml of quinidine (+ quinid). Scale bars represent 50 and 30 pA and 0.7 s for wild type and A116V-MiRP1 respectively. The inset represents the protocol: holding −80 mV, 1 s pulses from -60 to 20 mV in 10 mV steps, 2 s at −40 mV, 1 s interpulse interval. (B) Current-dose relationship for channels formed with wild type MiRP1 (open circles) and A116V-MiRP1 (filled squares) in the presence of increasing amounts of quinidine. Data from peak currents at −40 mV following a pre-pulse to +20 mV using the standard protocol. Theoretical lines were constructed to the function: 1/(1+H) were H=([drug]/Kᵢ)ⁿ. Kᵢ and n represent the equilibrium dissociation constant and the Hill coefficient, respectively. Fits give values for Kᵢ and n of 0.41±0.04 mg/L and 1.1±0.1 for wild type MiRP1 and 0.43±0.06 g/ml and 1.1±0.2 for A116V-MiRP1; six cells in each case.

FIG. 4: Channels formed with T8A-MiRP1 are more sensitive to sulfamethoxazole (SMX). (A) Current-dose relationship for channels formed with wild type MiRP1 (open circles) and T8A-MiRP1 (filled circles) in the presence of increasing amounts of TMP/SMX. Data from peak currents at −40 mV following a pre-pulse to +20 mV using the standard protocol. Fits give values for wild type MiRP1 of Kᵢ=0.380±0.040 mg/ml and n=1.8±0.3 and for T8A-MiRP1 of Kᵢ=0.210±0.030 mg/ml and n=1.9±0.2; 13 cells in each case. (B) Current-dose relationships in the presence of TMP for wild type MiRP1 (open circles) and T8A-MiRP1 (filled circles). Fits give values for Kᵢ and n of 0.071±0.012 mg/ml and 1.8±0.4 for wild type MiRP1 and 0.075±0.009 mg/ml and 1.9±0.3 for T8A-MiRP1; 5 cells each case. The data thus suggests inhibition was cooperative and that two SMX molecules were required to block. (C) Whole-cell currents recorded from channels formed with wild type MiRP1 (WT) and T8A-MiRP1 (T8A) in the absence (left column) or presence of 0.633 mg/ml of SMX (+SMX). Scale bars represent 50 pA and 40 pA for wild type and T8A, respectively, and 1 s. (D) Current-dose relationships in the presence of SMX for wild type MiRP1 (open circles) and T8A-MiRP1 (filled circles). Fits give values for Kᵢ and n of 2,600±200 mg/L and 1.6±0.6 for wild type MiRP1 and 0.675±0.060 mg/ml and 1.4±0.2 for T8A-MiRP1; 6 cells each case.

FIG. 5: Missense mutations and drug inhibition combine to diminish potassium flux. Simulated cardiac action potentials were employed (voltage ramp from 40 to −80 mV with a change in potential of −71 mV per s) to study channels formed with wild type MiRP1, A116V-MiRP1 and T8A-MiRP1 with HERG in CHO cells. (A) Whole-cell currents for simulated action potentials with wild type MiRP1 and A116V-MiRP1 (*) in the absence and presence of 500 ng/ml quinidine (+ quinid). Scale bars represent 1.3 pA/pF and 0.8 s. The inset represents the protocol: holding −80 mV, voltage ramp from 40 to −80 mV, dV/dt=−71 mV/s. (B) Normalized potassium flux assessed by measurement of area under the curve for simulated action potentials in cells expressing wild type MiRP1 (WT) or A116V-MiRP1 (A116V) in the presence of 0.5 mg/L quinidine (+ quinid) normalized to wild type channels without drug. Currents were divided by cell capacitance before normalization. Each bar represents the mean±SEM for 6-10 cells. (C) Whole-cell currents for simulated action potentials with wild type MiRP1 and T8A-MiRP1 (*) in the absence and presence of 0.300 mg/ml SMX. Scale bars represent 1.6 pA/pP and 0.8 s. (D) Normalized potassium flux assessed by measurement of area under the curve for simulated action potentials in cells expressing wild type MiRP1 (WT) and T8A-MiRP1 (T8A) in the presence of 0.300 mg/ml SMX normalized to wild type channels without drug. Currents were divided by the cell capacitance before normalization. Each bar represents the mean±SEM for 7-10 cells.

### DETAILED DESCRIPTION

MiRP1 Polypeptides and KCNE2 Genes

As used herein, the terms “MiRP1 polypeptide” or “KCNE2 gene” or “KCNE2 polynucleotide” refer to any mammalian MiRP1 protein or KCNE2 gene, and in particular, although not limited to, human MiRP1 proteins and KCNE2 genes. As described above, the gene encoding human MiRP1 (also referred to KCNE2) has been previously cloned and sequenced (See GenBank Accession No: AF071002). The terms “MiRP1 polypeptide” or “KCNE2 gene” however, are not limited to this specific sequence. For instance, the terms also refer to naturally occurring allelic variants and man-made substitution, insertion or deletion mutants that have a slightly different amino acid sequence than that specifically recited above.

As used herein, the family of proteins related to the human amino acid sequence of SEQ ID NO: 4 (also referred to as human wild-type MiRP1) refers to proteins that have been isolated from organisms in addition to humans. The methods used to identify and isolate other members of the family of proteins related to these proteins are readily available and known to persons skilled in the molecular biology field, including hybridization and sequence or homology screening methods.

As used herein, the terms “mutant MiRP1” and “mutant KCNE2 gene” and “KCNE2 polymorphism” refer to the protein or its encoding gene that is associated with a genetic predisposition to cardiac arrythmias such as acquired or antibiotic-induced LQTS.

As used herein, the term “drug-induced LQTS” or “antibiotic-induced LQTS” refers to a disorder or pathology where a “mutant MiRP1” or “mutant KCNE2 gene” or “KCNE2 polymorphism” is associated with drug- or antibiotic-induced pathological conditions such as cardiac arrythmias, including acquired LQTS.

As used herein, the term “MiRP1-mediated disease” refers to a disorder or pathology in which the presence of a “mutant MiRP1” or “mutant KCNE2 gene” or “KCNE2 polymorphism” is associated with or participates in a biological pathway in a manner that results in a pathological condition such as cardiac arrythmias.

As used herein, a polymorphism or mutation at position A116, refers to a mutant MiRP1 polypeptide containing a single amino acid alteration corresponding to alanine-116 of human MiRP1, as described by SEQ ID NO: 4, or a mutant KCNE2 gene with at least one sequence alteration in the nucleotide sequence corresponding to alanine-116 of human MiRP1, as described by SEQ ID NO: 3, that is associated with pathological conditions such as cardiac arrythmias, including acquired LQTS. Similarly, a polymorphism or mutation at position A116V, refers to a mutant MiRP1 polypeptide wherein the amino acid corresponding to alanine-116 of human MiRP1, as described by SEQ ID NO: 4, has been altered to valine or a mutant KCNE2 gene with at least one sequence alteration in the nucleotide sequence corresponding to alanine-116 of human MiRP1, as described by SEQ ID NO: 3, that replaces the alanine codon with one encoding valine, that is associated with pathological conditions such as cardiac arrythmias, including acquired LQTS.

As used herein, a polymorphism or mutation at position M54, refers to a mutant MiRP1 polypeptide containing a single amino acid alteration corresponding to methionine-54 of human MiRP1, as described by SEQ ID NO: 4, or a mutant KCNE2 gene with at least one sequence alteration in the nucleotide sequence corresponding to methionine-54 of human MiRP1, as described by SEQ ID NO: 3, that is associated with pathological conditions such as cardiac arrythmias, including acquired LQTS. Similarly, a polymorphism or mutation at position M54T, refers to a mutant MiRP1 polypeptide wherein the amino acid corresponding to methionine-54 of human MiRP1, as described by SEQ ID NO: 4, has been altered to threonine, or a mutant KCNE2 gene with at least one sequence alteration in the nucleotide sequence corresponding to methionine-54 of human MiRP1 as described by SEQ ID NO: 3, that replaces the methionine codon with one encoding threonine, that is associated with pathological conditions such as cardiac arrythmias, including acquired LQTS.

As used herein, a polymorphism or mutation at position I57, refers to a mutant MiRP1 polypeptide containing a single amino acid alteration corresponding to isoleucine-57 of human MiRP1, as described by SEQ ID NO: 4, or a mutant KCNE2 gene with at least one sequence alteration in the nucleotide sequence corresponding to isoleucine-57 of human MiRP1, as described by SEQ ID NO: 3, that is associated with pathological conditions such as cardiac arrythmias, including acquired LQTS. Similarly, a polymorphism or mutation at position I57T refers to a mutant MiRP1 polypeptide wherein the amino acid corresponding to isoleucine-57 of human MiRP1, as described by SEQ ID NO: 4, has been altered to threonine, or a mutant KCNE2 gene with at least one sequence alteration in the nucleotide sequence corresponding to isoleucine-57 of human M1, as described by SEQ ID NO: 3, that replaces the isoleucine codon with one encoding threonine, that is associated with pathological conditions such as cardiac arrythmias, including acquired LQTS.

As used herein, a polymorphism or mutation at position T8 refers to a mutant MiRP1 polypeptide containing a single amino acid alteration corresponding to threonine-8 of human MiRP1, as described by SEQ ID NO: 4, or a mutant KCNE2 gene with at least one sequence alteration in the nucleotide sequence corresponding to threonine-8 of human MiRP1, as described by SEQ ID NO: 3, that is associated with pathological conditions such as cardiac arrythmias, including acquired LQTS. Similarly, a polymorphism or mutation at position T8A, refers to a mutant MiRP1 polypeptide wherein the amino acid corresponding to threonine-8 of human MiRP1, as described by SEQ ID NO: 4, has been altered to alanine, or a mutant KCNE2 gene with at least one sequence alteration in the nucleotide sequence corresponding to threonine-8 of human MiRP1, as described by SEQ ID NO: 3, that replaces the threonine codon with one encoding alanine, that is associated with pathological conditions such as cardiac arrythmias, including acquired LQTS.

The proteins of the present invention are preferably in isolated form. As used herein, a protein is said to be isolated when physical, mechanical or chemical methods are employed to remove the protein from cellular constituents that are normally associated with the protein. A skilled artisan can readily employ standard purification methods to obtain such an isolated protein. MiRP1 proteins, or fragments thereof, may also be covalently modified by substitution, chemical enzymatic, or other appropriate means with a moiety other than a naturally occurring amino acid (for example, a detectable moiety such as an enzyme or radioisotope).

As used herein, a polynucleotide or nucleic acid molecule is said to be “isolated” when the nucleic acid molecule is substantially separated from and relative to contaminant or other nucleic acid molecules encoding other polypeptides with which the nucleic acids of the present invention are customarily associated. Nucleic acid molecules of the invention may be cloned into any available vector for replication and/or expression in suitable host cells. The host cells then may be used to recombinantly produce the encoded protein. Appropriate vectors, host cells and methods of expression are widely available.

Diagnostic Methods Using KCNE2 Polymorphisms

The invention provides a method for the diagnosis of MiRP1-mediated disease, such as drug- or antibiotic-induced LQTS, or a method of detecting a predisposition to MiRP1-mediated disease comprising the steps of detecting the presence or absence of a variant nucleotide at one or more of positions herein described in a patient sample and determining the status of the individual by reference to polymorphism in the KCNE2 gene. In preferred methods, a polymorphism is detected in the KCNE2 gene wherein the polymorphism is associated with cardiac arrythmia, acquired LQTS, drug-induced LQTS or antibiotic-induced LQTS.

In another preferred method, a polymorphism is detected at a position corresponding to nucleotides 95-97 in SEQ ID NO: 3 or at least one polymorphism is detected in a gene encoding a MiRP1 polypeptide at a position corresponding to amino acid 8 of SEQ ID NO: 4, wherein the presence of a polymorphism corresponds to an increased risk or genetic predisposition to cardiac arrythmia, including acquired LQTS. In another preferred embodiment, at least one polymorphism that results in the substitution of an alanine residue corresponding to position 8 in SEQ ID NO: 4 is detected, wherein the absence of a threonine, for instance the presence of an alanine, is associated with an increased risk or genetic predisposition to LQTS, and the presence of a threonine residue is associated with decreased risk.

In another preferred method, a polymorphism is detected at a position corresponding to nucleotide 233-235 in SEQ ID NO: 3 or at least one polymorphism is detected in a gene encoding a MiRP1 polypeptide at a position corresponding to amino acid 54 of SEQ ID NO: 4, wherein the presence of a polymorphism corresponds to an increased risk or genetic predisposition to cardiac arrythmia, including acquired LQTS. In another preferred embodiment, at least one polymorphism that results in the substitution of a methionine residue corresponding to position 54 in SEQ ID NO: 4 is detected, wherein the absence of a methionine, for instance the presence of a threonine, is associated with an increased risk or genetic predisposition to LQTS, and the presence of a methionine residue is associated with decreased risk.

In another preferred method, a polymorphism is detected at a position corresponding to nucleotide 242-244 in SEQ ID NO: 3 or at least one polymorphism is detected in a gene encoding a MiRP1 polypeptide at a position corresponding to amino acid 57 of SEQ ID NO: 4, wherein the presence of a polymorphism corresponds to an increased risk or genetic predisposition to cardiac arrythmia, including acquired LQTS. In another preferred embodiment, at least one polymorphism that results in the substitution of an isoleucine residue corresponding to position 57 in SEQ ID NO: 4 is detected, wherein the absence of a isoleucine, for instance the presence of a threonine, is associated with an increased risk or genetic predisposition to LQTS, and the presence of a isoleucine residue is associated with decreased risk.

In another preferred method, a polymorphism is detected at a position corresponding to nucleotide 419-421 in SEQ ID NO: 4 or at least one polymorphism is detected in a gene encoding a MiRP1 polypeptide at a position corresponding to amino acid 116 of SEQ ID NO: 3, wherein the presence of a polymorphism corresponds to an increased risk or genetic predisposition to cardiac arrythmia, including acquired LQTS. In another preferred embodiment, at least one polymorphism that results in the substitution of an alanine residue corresponding to position 116 in SEQ ID NO: 3 is detected, wherein the absence of an alanine, for instance the presence of a valine is associated with an increased risk or genetic predisposition to LQTS, and the presence of an alanine residue is associated with decreased risk.

Any sample comprising cells or nucleic acids from the patient or subject to be tested may be used. Preferred samples are those easily obtained from the patient or subject. Such samples include, but are not limited to blood, peripheral lymphocytes, epithelia cell swabs, bronchoalveolar lavage fluid, sputum, or other body fluid or tissue obtained from an individual. It will be appreciated that the test sample may comprise KCNE2 nucleic acid that has been amplified using any convenient technique, e.g., PCR before analysis of allelic variation. As described below, any available means of detecting a sequence polymorphism(s) of the invention may be used in the methods.

In another method of the invention, the diagnostic methods described herein are used in the development of new drug therapies, which selectively target one or more KCNE2 polymorphisms that are associated with cardiac arrythmias, LQTS and specifically antibiotic-induced LQTS. In one format, the diagnostic assays of the invention may be used to stratify patient populations by separating out patients with a genetic predisposition to a specific medical disorder from the general population. Identification of a link between polymorphisms and predisposition to disease development or response to drug therapy may have a significant impact on the design of new drugs by assisting in the analysis of a drugs efficacy or effects on specific populations of patients. For instance, drugs may be designed to regulate the biological activity of variants implicated in the disease process while minimizing effects on other variants.

Gene expression or activity may also be used to track or predict the progress or efficacy of a treatment regime in a patient. For instance, a patient's progress or response to a given drug may be monitored by measuring variant gene expression in a tissue or cell sample after treatment or administration of the drug. Variant gene expression in the post-treatment sample may then be compared to gene expression from tissue or cells from the same patient before treatment.

Detection of Polymorphisms

As described above, detection of the KCNE2 polymorphisms of the invention generally comprises the step of determining the sequence of a KCNE2 gene in a sample, preferably a patient sample, at one or more of the positions herein described.

Any analytical procedure may be used to detect the presence or absence of variant nucleotides at one or more polymorphic positions of the invention. In general, the detection of allelic variation requires a mutation discrimination technique, optionally an amplification reaction and optionally a signal generation system. Many current methods for the detection of allelic variation are reviewed by Nollau et. al. (1997) Clin. Chem. 43, 1114-1120; and in standard textbooks, for example, Landegren (1996) Laboratory Protocols for Mutation Detection, Oxford University Press; and Newton & Graham (1997) PCR (2nd edition) BIOS Scientific Publishers. Any means of mutation detection or discrimination may be used. For instance DNA sequencing, scanning methods, hybridization, extension-based methods, incorporation-based methods, restriction enzyme-based methods and ligation-based methods may be used in the methods of the invention. Sequencing methods include, but are not limited to, direct sequencing and sequencing by hybridization. Scanning methods include, but are not limited to, protein truncation test (PTT), single-strand conformation polymorphism analysis (SSCP), denaturing gradient gel electrophoresis (DGGE), temperature gradient gel electrophoresis (TGGE), cleavage, heteroduplex analysis, chemical mismatch cleavage (CMC), and enzymatic mismatch cleavage.

Hybridization-based methods of detection include, but are not limited to, solid phase hybridization such as dot blots, multiple allele specific diagnostic assay (MASDA), and reverse dot blots, oligonucleotide arrays (DNA Chips). Solution phase hybridization methods may also be used, such as Taqman.

Extension based methods include, but are not limited to, amplification refractory mutation system (ARMS), amplification refractory mutation system linear extension 5 (ALEX), and competitive oligonucleotide priming system (COPS).

Incorporation-based detection methods include, but are not limited to, minisequencing and arrayed primer extension (APEX). Restriction enzyme-based detection systems include, but are not limited to, RFLP, and restriction site generating PCR. Lastly, ligation based detection methods include, but are not limited to, oligonucleotide ligation assay (OLA).

Signal generation or detection systems that may be used in the methods of the invention include, but are not limited to, fluorescence methods such as fluorescence resonance energy transfer (FRET), fluorescence quenching, fluorescence polarization as well as other chemiluminescence, electrochemiluminescence, Raman, radioactivity, colorimetric methods, hybridization protection assay and mass spectrometry.

Further amplification methods include, but are not limited to self sustained replication (SSR), nucleic acid sequence based amplification (NASBA), ligase chain reaction (LCR), strand displacement amplification (SDA) and branched DNA (b-DNA).

Nucleotide Primers and Probes

The invention further provides nucleotide primers, which can detect the polymorphisms of the invention. In one embodiment of the invention, primers are prepared that are capable of detecting a KCNE2 gene polymorphism at one or more of the positions herein described. Preferred primers allow detection of a KCNE2 gene polymorphism associated with cardiac arrythmias, LQTS, and specifically antibiotic-induced LQTS, such as a polymorphism at nucleotides 95-97, 233-235, 242-244 or 419-421, corresponding to the luna KCNEZ gene (SEQ ID NO:3).

Allele specific primers are typically used together with a constant primer, in an amplification reaction such as a PCR reaction, which provides the discrimination between alleles through selective amplification of one allele at a particular sequence position. The allele specific primer is preferably about 10, 12, 15, 17, 19 or up to about 100 or more nucleotides in length, more preferably about 17-35 nucleotides in length, and more preferably about 10-30 nucleotides in length.

The allele specific primer preferably corresponds exactly with the allele to be detected but allele specific primers may be derivatives wherein about 6-8 of the nucleotides at the 3′ terminus correspond with the Allele to be detected and wherein up to 10, such as up to 8, 6, 4, 2, or 1 of the remaining nucleotides may be varied without significantly affecting the properties of the primer.

Primers may be manufactured using any convenient method of synthesis. Examples of such methods may be found in standard textbooks, for example: Agrawal (1993) Protocols for Oligonucleotides and Analogues; Synthesis and Properties, Methods in Molecular Biology Series, Humana Press. If required, the primers may be labeled to facilitate detection.

The invention also provides allele-specific probes that are capable of detecting KCNE2 gene polymorphisms associated with antibiotic-induced LQTS. Preferred probes allow detection of a KCNE2 gene polymorphism associated antibiotic-induced LQTS, such as a polymorphism nucleotides 95-97, 233-235, 242-244, or 419-421, corresponding to the KCNE2 gene (SEQ ID NO: 3).

Such probes are of any convenient length, such as up to about 100 bases or more, up to 50 bases, and more conveniently up to 30 bases in length, such as for example 8-25 or 8-15 bases in length. In general such probes will comprise base sequences entirely complementary to the corresponding wild-type or variant locus in the gene. However, if required, one or more mismatches may be introduced, provided that the discriminatory power of the oligonucleotide probe is not unduly affected. The probes of the invention may carry one or more labels to facilitate detection.

According to another aspect of the present invention there is provided a diagnostic kit comprising an allele specific oligonucleotide probe or primer of the invention and/or an allele-nonspecific primer of the invention. The diagnostic kits may comprise appropriate packaging and instructions for use in the methods of the invention. Such kits may further comprise appropriate buffer(s), nucleotides, and polymerase(s) such as thermostable polymerases, for example Taq polymerase.

The present invention also includes a computer readable medium comprising at least one novel polynucleotide sequence of the invention stored on the medium, such as a nucleotide sequence spanning a polymorphism in KCNE2 gene as herein described. The computer readable medium may be used, for example, in homology searching, mapping, haplotyping, genotyping or pharmacogenetic analysis or any other bioinformatic analysis.

The polynucleotide sequences of the invention, or parts thereof, particularly those relating to and identifying the single nucleotide polymorphisms identified herein represent a valuable information source, for example, to characterize individuals in terms of haplotype and other sub-groupings, such as investigating the susceptibility to treatment with particular drugs. These approaches are most easily facilitated by storing the sequence information in a computer readable medium and then using the information in standard bioinformatics programs or to search sequence databases using state of the art searching tools such as GCG Wisconsin Package (Accelrys Inc.). Thus, the polynucleotide sequences of the invention are particularly useful as components in databases useful for sequence identity and other search analyses. As used herein, storage of the sequence information in a computer readable medium and use in sequence databases in relation to “polynucleotide”, or “polynucleotide sequence of the invention” covers any detectable chemical or physical characteristic of a polynucleotide of the invention that may be reduced to, converted into or stored in a tangible medium, such as a computer disk, preferably in a computer readable form. For example, chromatographic scan data or peak data, photographic scan or peak data, mass spectrographic data, sequence gel (or other) data may be included.

A computer based method is also provided for performing sequence identification, said method comprising the steps of providing a polynucleotide sequence comprising a polymorphism of the invention in a computer readable medium; and comparing said polymorphism containing polynucleotide sequence to at least one other polynucleotide or polypeptide sequence to identify identity (homology), i.e., screen for the presence a polymorphism.

Methods to Identify Agents that Modulate Expression

Another embodiment of the present invention provides methods for identifying agents that modulate the expression of a nucleic acid encoding a KCNE2 polymorphism of the invention. Such assays may utilize any available means of monitoring for changes in the expression level of the nucleic acids of the invention. As used herein, an agent is said to modulate the expression of a nucleic acid of the invention if it is capable of up- or downregulating expression of the nucleic acid in a cell.

In one assay format, the expression of a nucleic acid encoding a KCNE2 polymorphism of the invention in a cell or tissue sample is monitored directly by hybridization to the nucleic acids of the invention. Cell lines or tissues are exposed to the agent to be tested under appropriate conditions and time and total RNA or mRNA is isolated by standard procedures such those disclosed in Sambrook et al. (1989) Molecular Cloning—A Laboratory Manual, Cold Spring Harbor Laboratory Press.

Probes to detect differences in RNA expression levels between cells exposed to the agent and control cells may be prepared as described above. Hybridization conditions are modified using known methods, such as those described by Sambrook et al. and Ausubel et al. as required for each probe. Hybridization of total cellular RNA or RNA enriched for polyadenylated (polyA) RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a silicon chip or a porous glass wafer. The chip or wafer can then be exposed to total cellular RNA or polyA-RNA from a sample under conditions in which the affixed sequences will specifically hybridize to the RNA. By examining for the ability of a given probe to specifically hybridize to an RNA sample from an untreated cell population and from a cell population exposed to the agent, agents which up- or down-regulate expression are identified.

Methods to Identify Agents that Modulate Activity

Another embodiment of the present invention provides methods for identifying agents that modulate the cellular level or concentration or at least one activity of a variant protein of the invention. Such methods or assays may utilize any means of monitoring or detecting the desired activity.

In one format, the relative amounts of a protein of the invention between a cell population that has been exposed to the agent to be tested compared to an unexposed control cell population may be assayed. In this format, probes such as specific antibodies are used to monitor the differential expression of the protein in the different cell populations. Cell lines or populations are exposed to the agent to be tested under appropriate conditions and time. Cellular lysates may be prepared from the exposed cell line or population and a control, unexposed cell line or population. The cellular lysates are then analyzed with the probe.

Antibody probes are prepared by immunizing suitable mammalian hosts in appropriate immunization protocols using the peptides, polypeptides or proteins of the invention if they are of sufficient length, or, if desired, or if required to enhance immunogenicity, conjugated to suitable carriers. Methods for preparing immunogenic conjugates with carriers such as BSA, KLH or other carrier proteins are well known in the art. In some circumstances, direct conjugation using, for example, carbodiimide reagents may be effective; in other instances linking reagents such as those supplied by Pierce Chemical Company, may be desirable to provide accessibility to the hapten. The hapten peptides can be extended at either the amino or carboxy terminus with a cysteine residue or interspersed with cysteine residues, for example, to facilitate lining to a carrier. Administration of the immunogens is conducted generally by injection over a suitable time period and with use of suitable adjuvants, as is generally understood in the art. During the immunization schedule, titers of antibodies are taken to determine adequacy of antibody formation. While the polyclonal antisera produced in this way may be satisfactory for some applications, for pharmaceutical compositions, use of monoclonal preparations is preferred.

Immortalized cell lines which secrete the desired monoclonal antibodies may be prepared using the standard method of Kohler & Milstein (1975) Nature 256, 495-497 or modifications which effect immortalization of lymphocytes or spleen cells, as is generally known in the art. The immortalized cell lines secreting the desired antibodies are screened by immunoassay in which the antigen is the peptide hapten polypeptide or protein. When the appropriate immortalized cell culture secreting the desired antibody is identified, the cells can be cultured either in vitro or by production in ascites fluid.

The desired monoclonal antibodies are then recovered from the culture supernatant or from the ascites supernatant. Fragments of the monoclonals or the polyclonal antisera which contain the immunologically significant portion can be used as antagonists, as well as the intact antibodies. Use of immunologically reactive fragments, such as the Fab, Fab′ of F(ab′)₂ fragments is often preferable, especially in a therapeutic context, as these fragments are generally less immunogenic than the whole immunoglobulin.

The antibodies or fragments may also be produced, using current technology, by recombinant means. Antibody regions that bind specifically to the desired regions of the protein can also be produced in the context of chimeras with multiple species origin, such as humanized antibodies.

Agents that are assayed in the above methods can be randomly selected or rationally selected or designed. As used herein, an agent is said to be randomly selected when the agent is chosen randomly without considering the specific sequences involved in the association of a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a nonrandom basis which takes into account the sequence of the target site and/or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any functional consensus site.

The agents of the present invention can be, as examples, peptides, small molecules, vitamin derivatives, nucleic acid molecules such as antisense molecules that specifically recognize a mutant MiRP1 as well as carbohydrates. Dominant negative proteins, DNAs encoding these proteins, antibodies to these proteins, peptide fragments of these proteins or mimics of these proteins may be introduced into cells to affect function. “Mimic” as used herein refers to the modification of a region or several regions of a peptide molecule to provide a structure chemically different from the parent peptide but topographically and functionally similar to the parent peptide (see e.g., Meyers (1995) Molecular Biology and Biotechnology VCH Publishers, pp. 659-664). A skilled artisan can readily recognize that there is no limit as to the structural nature of the agents of the present invention.

The peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

Transgenic Animals

Transgenic animals containing mutant, knock-out or modified genes corresponding to the cDNA sequences of SEQ ID NO: 1, SEQ ID NO: 5, SEQ ID NO: 7 or SEQ ID NO: 9 are also included in the invention. Transgenic animals are genetically modified animals into which recombinant, exogenous or cloned genetic material has been experimentally transferred. Such genetic material is often referred to as a transgene. The nucleic acid sequence of the transgene, in this case a form of SEQ ED NO: 1, SEQ ID NO: 5, SEQ ID NO: 7 or SEQ ID NO: 9 may be integrated either at a locus of a genome where that particular nucleic acid sequence is not otherwise normally found or at the normal locus for the transgene. The transgene may consist of nucleic acid sequences derived from the genome of the same species or of a different species than the species of the target animal.

The term “germ cell line transgenic animal” refers to a transgenic animal in which the genetic alteration or genetic information was introduced into a germ line cell, thereby conferring the ability of the transgenic animal to transfer the genetic information to offspring. If such offspring in fact possess some or all of that alteration or genetic information, then they too are transgenic animals. The alteration or genetic information may be foreign to the species of animal to which the recipient belongs, foreign only to the particular individual recipient, or may be genetic information already possessed by the recipient. In the last case, the altered or introduced gene may be expressed differently than the native gene.

Transgenic animals can be produced by a variety of different methods including transfection, electroporation, microinjection, gene targeting in embryonic stem cells and recombinant viral and retroviral infection (see, e.g., U.S. Pat. Nos. 4,736,866 & 5,602,307; Mullins et al. (1993) Hypertension 22, 630-633; Brenin et al. (1997) Surg. Oncol. 6, 99-110; Tuan (1997), Recombinant Gene Expression Protocols, Humana Press).

A number of recombinant or transgenic mice have been produced, including those which express an activated oncogene sequence (U.S. Pat. No. 4,736,866); express simian SV 40 T-antigen (U.S. Pat. No. 5,728,915); lack the expression of interferon regulatory factor 1 (IRF-1) (U.S. Pat. No. 5,731,490); exhibit dopaminergic dysfunction (U.S. Pat. No. 5,723,719); express at least one human gene which participates in blood pressure control (U.S. Pat. No. 5,731,489); display greater similarity to the conditions existing in naturally occurring Alzheimer's disease (U.S. Pat. No. 5,720,936); have a reduced capacity to mediate cellular adhesion (U.S. Pat. No. 5,602,307); possess a bovine growth hormone gene (Clutter et al. (1996) Genetics 143, 1753-1760); or, are capable of generating a fully human antibody response (McCarthy (1997) Lancet 349, 405-406).

While mice and rats remain the animals of choice for most transgenic experimentation, in some instances it is preferable or even necessary to use alternative animal species. Transgenic procedures have been successfully utilized in a variety of non-murine animals, including sheep, goats, pigs, dogs, cats, monkeys, chimpanzees, hamsters, rabbits, cows and guinea pigs (see, e.g., Kim et al. (1997) Mol. Reprod. Dev. 46, 515-526; Houdebine (1995) Reprod. Nutr. Dev. 35, 609-617; Petters (1994) Reprod. Fertil. Dev. 6, 643-645; Schnieke et al. (1997) Science 278, 2130-2133; and Amoah (1997) J. Animal Science 75, 578-585). The method of introduction of nucleic acid fragments into recombination competent mammalian cells can be by any method which favors co-transformation of multiple nucleic acid molecules. Detailed procedures for producing transgenic animals are readily available to one skilled in the art, including the disclosures in U.S. Pat. Nos. 5,489,743 & 5,602,307.

Without further description, it is believed that one of ordinary skill in the art can, using the preceding description and the following illustrative examples, make and utilize the compounds of the present invention and practice the claimed methods. The following working examples therefore, specifically point out preferred embodiments of the present invention, and are not to be construed as limiting in any way the remainder of the disclosure.

### EXAMPLES

### Example 1

Mutation Analysis

The GenBank accession number for human KCNE2 encoding MiRP1 is A17071002. Genomic DNA was isolated from peripheral blood leukocytes by standard methods and two overlapping regions of the KCNE2 coding sequence screened for the presence of nucleotide sequence polymorphism by SSCP analysis (Orita et al. (1989) Proc. Natl. Acad. Sci. USA 86, 2766-2770).

Oligonucleotide primer pairs used to amplify the KCNE2 coding sequence were: section I (nucleotides 32-259 of AF071002), forward primer: 5′-tgt tcg cct att tta tta ttt-3′ (SEQ ID NO: 11); reverse primer: 5′-aaa gag aac att cca atc at-3′ (SEQ ID NO: 12); section II (corresponding to nucleotides 212471), forward primer: 5′-tac tat gtc atc ctg tac ctc-3′ (SEQ ID NO: 13); reverse primer: 5′-gtt agc ttg gtg cct ttc t-3′ (SEQ ID NO: 14). Amplification reactions were carried out using 200 ng genomic DNA, 500 nM primers, 200 nM deoxynucleotide triphosphates and Taq polymerase. SSCP analysis was performed on 0.5× MDE gels subjected to overnight electrophoresis at 4 watts and stained with silver nitrate. Abnormal conformers were excised from dried gels, eluted into sterile water at room temperature, re-amplified using the original primers, and sequenced using dye terminator chemistry. The presence of the KCNE2-A116V polymorphism was confirmed independently on PCR amplified genomic DNA using an allele-specific hybridization assay with a [³²P] end-labeled primer: 5′-aac att ggt gtg gct ggg tt-3′ (SEQ ID NO: 15).

### Example 2

Heterologous Expression

Mutations identified in patients were produced in wild-type human KCNE2 in pGA1 (Abbott et al. (1999) Cell 97, 175-187) by pfu-based mutagenesis (QuickChange Kit; Stratagene) followed by insertion of mutant gene fragments into translationally-silent restriction sites in the wild type construct and confirmed by automated DNA sequencing. Transient transfection of CHO cells was by DEA Dextan, Chloroquine and DMSO shock (Abbott et al. (1999) Cell 97, 175-187).

### Example 3

Electrophysiology

Data were recorded using an Axopatch 200 B amplifier (Axon Instruments), a Quadra 800 Macintosh computer and Pulse software. Sampled at 1 kHz, data are presented as mean±SEM. All experiments were performed at room temperature. Holding voltage was −80 mV. Standard voltage protocol: 1-s pre-pulse from −60 to 20 mV in 10 mV step followed by 2-s step to 40 mV with a 3-s interpulse interval. Simulated cardiac action potential protocol: voltage ramp from 40 to −90 mV with a rate of change of −71 mV per second. Data are presented after ohmic leak subtraction (standard protocol) or subtraction of residual current with after full drug block (simulated action potential). If leak was greater than 25% of total current, cells were discarded. The bath solution was (in mM): 100 mM NaCl, 4 mM KCl, 1 mM calcium chloride, 0.7 mM magnesium chloride and 10 mM HEPES (pH 7.5) with NaOH. The pipette solution was: 100 mM KCl, 10 mM HEPES, 1 mM magnesium chloride, 2 mM EGTA (pH 7.5) with KOH. Stock solutions of oxatomide (Jensen), quinidine (Sigma) and procainamide (Sigma) were prepared by dissolution in bath solution at concentrations of 0.01, 1 and 10 mM, respectively. Oxatomide solution were prepared fresh daily. Sulfamethoxazole and trimethoprim (Sigma) are poorly soluble in water and were dissolved in ethanol at 100 mM and methanol at 20 mM, respectively. Solutions were then prepared daily by diluting stocks in bath solution. Sulfamethoxazole and trimethoprim were mixed 1:5 by weight as in TMP/SMX; control solutions for these studies were supplemented with equal levels of ethanol and/or methanol.

### Example 4

KCNE2 Polymorphisms Associated With Drug-Induced LQTS.

Genomic DNA samples were obtained from 98 patients who experienced excessive prolongation of the QT interval (>600 msec) or developed TdP during drug treatment. As KCNE2 consists of a single coding exon (encoding a peptide of just 123 amino acids), it was readily amplified in two overlapping segments from genomic DNA allowing the coding region to be screened for sequence polymorphism. Three sporadic mutations and one common polymorphism were found in four patients in our population. Mutations were identified that encoded M54T- MiRP1, I57T-MiRP1 and A116V-MiRP1; the first two were previously identified in patients with sporadic LQTS (Abbott et al. (1999) Cell 97, 175-187). One patient carried a polymorphism producing T8A-MiRP1, a KCNE2 variant seen previously in 16 of 1,010 control individuals (Abbott et al. (1999) Cell 97, 175-187). The positions of these residues relative to the cell membrane are indicated on a schematic rendition of the predicted topology of MiRP1 (Davies et al. (1998) Pharmatherapeutica 3, 365-369). The KCNE2 mutation that substituted threonine for methionine at amino acid 54 (M54T) was found in a patient who presented with procainamide-induced LQTS. This mutation was seen once before among 230 patients with sporadic LQTS but not in 1,010 controls (Abbott et al. (1999) Cell 97, 175-187); the antiarrhythmic medication procainamide has been associated with acquired LQTS previously (Roden et al. (1996) Circulation 94, 1996-2012).

The missense mutation that substituted a threonine for isoleucine at residue 57 (157T) was found in a patient with a prolonged QT interval induced by the histamine H1 receptor antagonist oxatomide. This mutation was seen once before among 230 patients with sporadic LQTS (Abbott et al. (1999) Cell 97, 175-187); oxatomide has not previously been associated with dysrrhythmia.

A novel missense mutation substituting valine for alanine at position 116 (A116V) was found in a 55 year-old white female with a history of cardiac arrest associated with cocaine and alcohol abuse. Subsequent treatment with quinidine and mexiletine was tolerated for six years. After mexiletine was discontinued, she had syncope with TdP while on quinidine alone. Evaluation at that time also revealed new onset heart failure. Sequence analysis revealed a C to T transition that was not detected in 1,010 control individuals evaluated previously (Abbott et al. (1999) Cell 97, 175-187) or 200 control genes assessed here by an allele specific hybridization assay. The antiarrhythmic quinidine has been associated with acquired LQTS (Roden et al. (1996) Circulation 94, 1996-2012).

A fourth missense mutation was identified in a 45-year-old white male with a history of Marfan syndrome who had a normal QT interval at baseline but developed marked QT prolongation after three oral doses of TMP/SMX for treatment of a minor infection. Screening revealed a polymorphism that substituted alanine for threonine at MiRP1 position 8 (T8A). This KCNE2 polymorphism was previously seen in 16 of 1,010 control individuals, 1 of 230 patients with sporadic LQTS, and 1 patient with quinidine-induced dysrrhythmia among 20 individuals with drug-induced arrhythmia (Abbott et al. (1999) Cell 97, 175-187).

### Example 5

Ion Channels Comprising Mutant MiRP1 Polypeptides.

Wild-type and mutant MiRP1/HERG channels were studied by transient expression in Chinese Hamster Ovary (CHO) cells using the whole-cell patch clamp configuration as previously described (Abbott et al. (1999) Cell 97, 175-187). The three sporadic mutations M54T-MiRP1, I57T- MiRP1 and A116V-MiRP1 produced reductions of 34 to 47% in current density at 40 mV (that is, whole-cell current divided by membrane capacitance to normalize for cell size) while only a 15% decrease was observed with T8A-MiRP1. This was compatible with our prior study showing that channels with M54T-MiRP1 activated less readily and deactivated more rapidly than channels formed with either wild type or T8A-MiRP1. Consistent with these observations, patients carrying M54T-MiRP1 showed slightly prolonged QT intervals in the drug-free state while the patient with T8A-MiRP1, like 17/18 individuals with this genotype studied previously, had a normal QT interval at baseline.

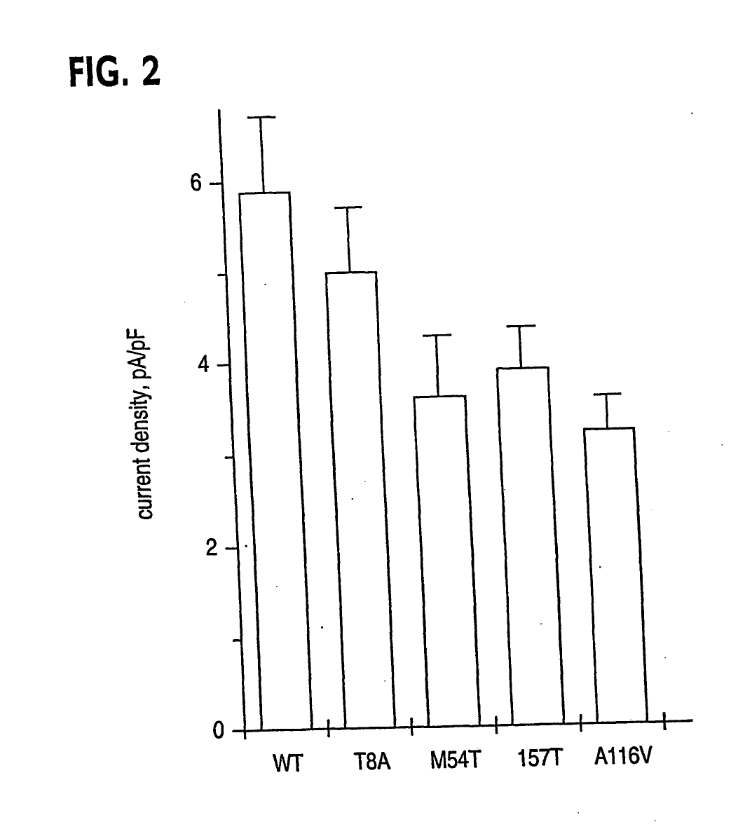

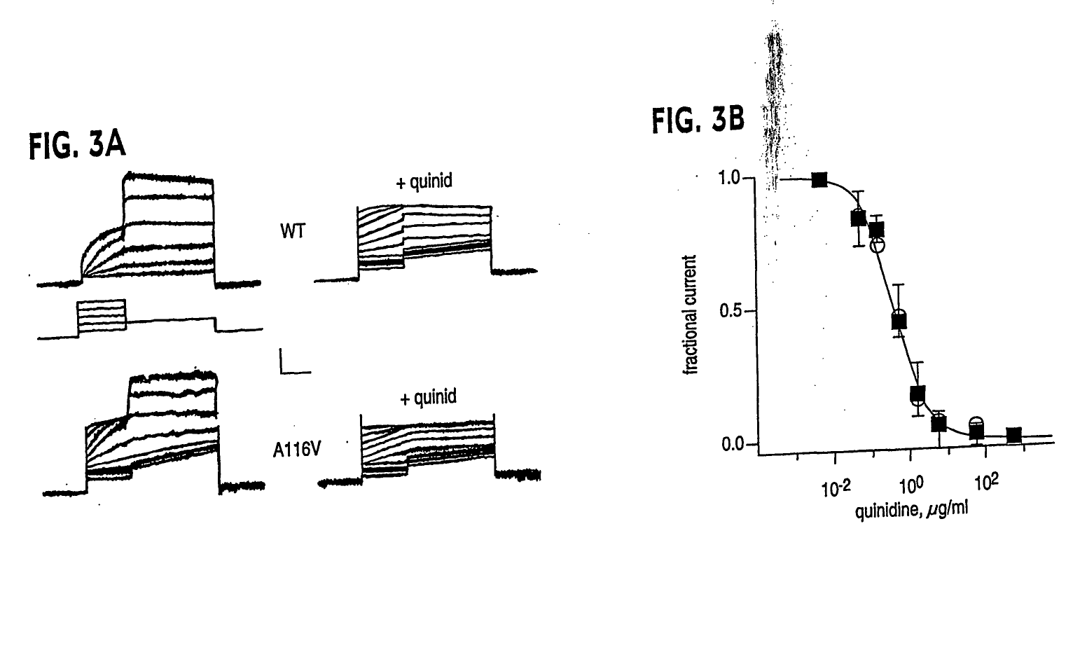

The three sporadic mutations identified in our patients with drug-induced LQTS did not alter sensitivity to drug inhibition FIG. 3A shows currents carried by channels formed with wild type MiRP1 or A116V-MiRP1 in the absence and presence of quinidine; the two channels showed the same sensitivity to equilibrium blockade (FIG. 2). Similarly, channels with wild-type MiRP1 or M54T-MiRP1 showed the same sensitivity to procainamide (with equilibrium inhibition constants, Kᵢ of 0.190±0.030 and 0.185±0.035 mg/ml (n=6), respectively) while those with wild type MiRP1 and I57T-MiRP1 had the same sensitivity to oxatomide Kᵢ=180±50 and 800±40 ng/ml (n=5), respectively). Of note, levels of quinidine and oxatomide that achieved half-maximal inhibition of channels in experimental cells were close to their therapeutic concentrations in the plasma of patients. In contrast, procainamide and its principle metabolite N-acetylprocainamide achieved half-maximal blockade at levels over fifty-fold higher than those found in plasmae; the metabolite was even less potent than the parent compound Kᵢ=0.400±0.060 and 0.390∓0.070 mg/ml for wild-type MiRP1 and M54T-MiRP1 (n=5), respectively).

### Example 6

Ion Channels Comprising T8A-MiRP1 Polypeptides.

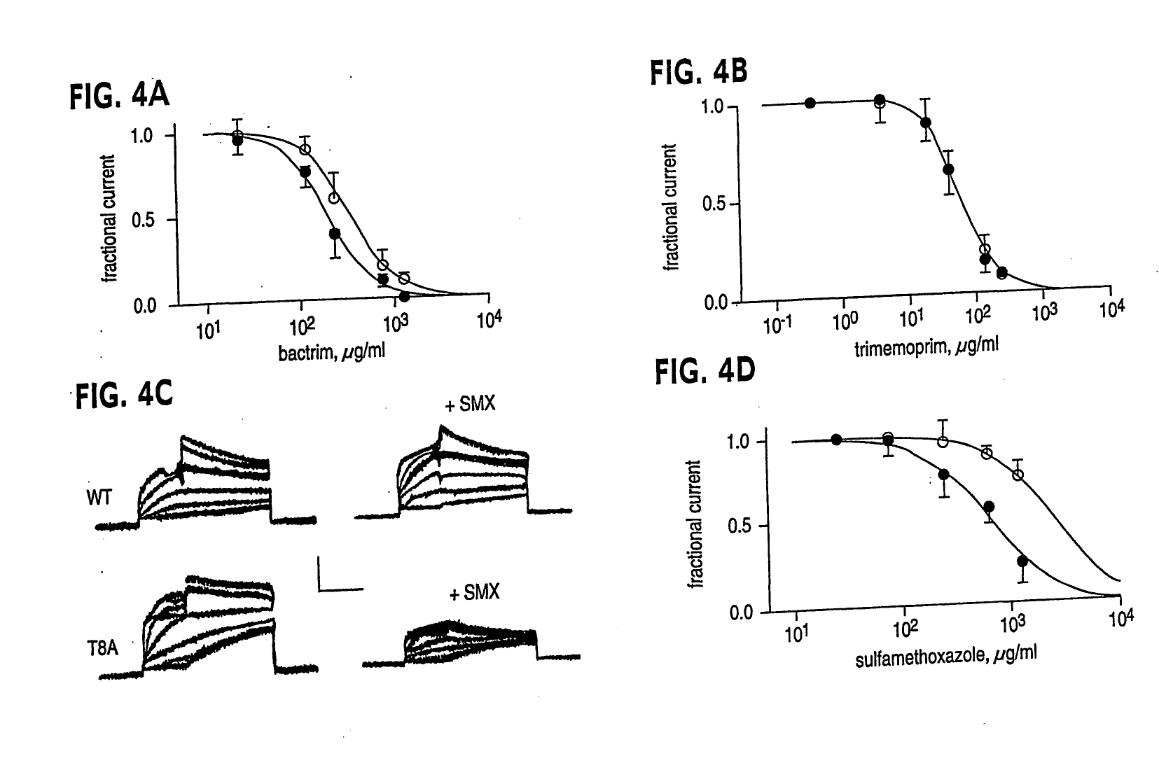

Wild-type and T8A-MiRP1 channels were studied with TMP/SMX as this agent was associated with a prolonged QT interval in our patient (FIG. 4). While both channel types were blocked, those with T8A-MiRP1 were about two-fold more sensitive to TMP/SMX (FIG. 4A). Each active compound, TMP and SMX, was then investigated separately. FIG. 3B shows that TMP inhibited both channels with similar affinity (Kᵢ of about 0.075 mg/ml); a level of the agent far in excess of its usual plasma level, about 0.005 mg/ml. In contrast, SMX (0.633 mg/ml) had almost no effect on wild type channels but inhibited more than half the flux through channels formed with T8A-MiRP1 at 40 mV FIG. 4C). Channels with T8A-MiRP1 were at least four-fold more sensitive to SMX than wild type (FIG. 4D). It seems reasonable to ascribe the pro-arrhytmic effects of Bactrim in our patient to its SMX component as serum levels for this agent can exceed 0.300 mg/ml.

A notable difference in the effect of SMX on channels formed with (T8A) and wild-type MiRP1 was apparent when gating kinetics were evaluated; the drug speeded deactivation (closure) only of channels with the SNP (Table 1). Thus, SMX increased both the fast and slow deactivation time constants for T8A-MiRP1 channels (enhancing τ_{fast} and τ_{slow} about four-fold and two-fold, respectively) but had no significant effect on wild-type channels. As before, (see e.g., Abbott et al. (1999) Cell 97, 175-187) channels with the single nucleotide polymorphism showed a small shift in their half-maximal activation voltage (V_{1/2} but no change in slope factor Vₛ) compared to wild type (Table 1); SMX had no significant effect on the V_{1/2} or Vₛ of either channel (Table 1).

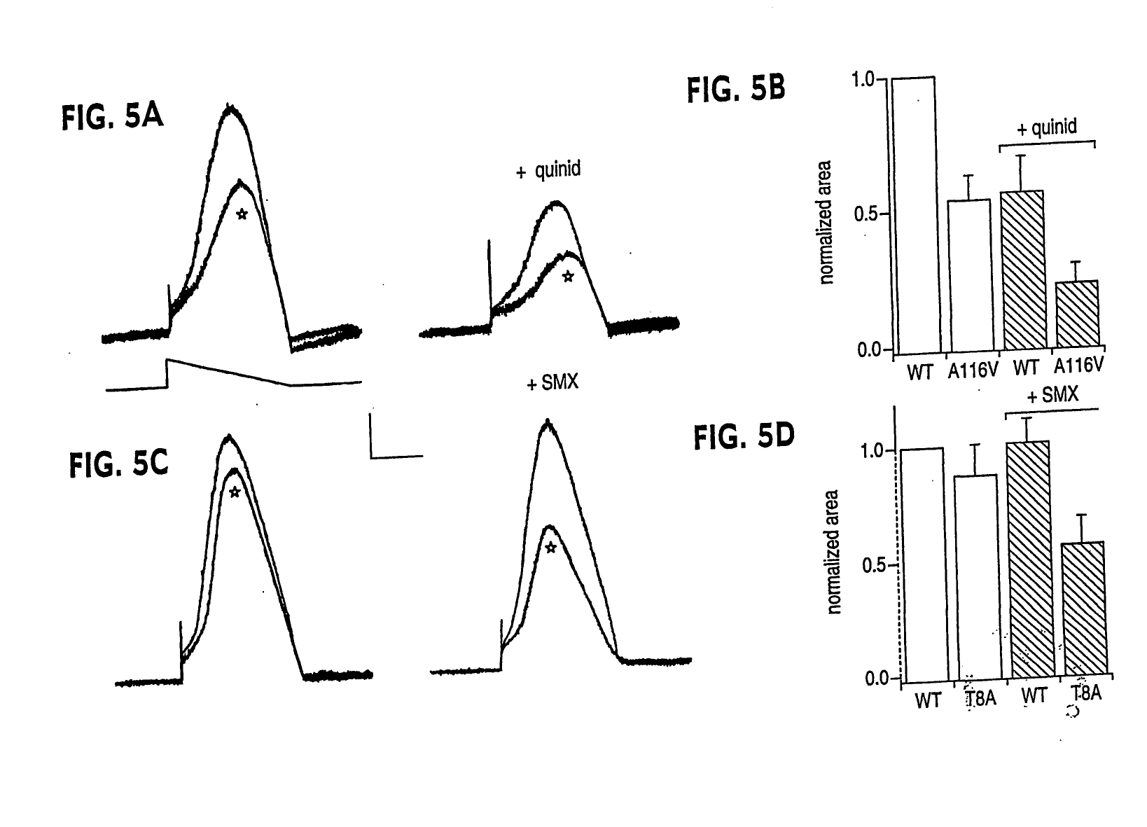

To approximate the in vivo case with experimental cells, a voltage protocol that mimics a cardiac action potent the presence of physiological levels of drugs associated with LQTS in our patients was employted. Flux of potassium ions across the membrane in each cycle was estimated from the area under the current curve. FIG. 5A shows raw traces of currents passed by wild-type MiRP1 or A116V-MiRP1 channels in the absence and presence of 500 ng/ml quinidine. At baseline, the mutant channels pass 46±6% less potassium than wild-type (n=5). The two channel types were similarly sensitive to drug inhibition showing about 50% block at this drug dose. Therefore, with drug, A116V MiRP1 channels passed about one-quarter of the current carried by unblocked wild-type channels. These findings support the notion that A116V-MiRP1 decreases the ability of the myocardium to repolarize at baseline, thereby placing such patients at increased risk for drug-induced arrhythmia when subsequently exposed to quinidine at therapeutic levels. In this case, superimposed heart failure further reduced repolarization reserve thereby to increase the likelihood of TdP. FIG. 5C shows raw traces of currents passed by wild type MiRP1 and T8A-MiRP1 channels in the absence and presence of 0.300 mg/ml SMX. In the absence of drug, channels formed with the T8A-MiRP1 were largely indistinguishable from wild-type MiRP1, as expected (Table 1). While SMX at this dose did not significantly affect wild-type channels, current through T8A-MiRP1 channels was reduced to half of the wild-type level (FIG. 5D) (n=5). Concordant with the observation that SMX speeds deactivation of T8A-MiRP1 channels, peak current was reached more rapidly in the presence of the drug (FIG. 5D), presumably due to increased channel availability, as with a disease-associated mutation of HERG that speeds deactivation.

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety.

### APPENDIX A

TABLE 1
Gating parameters for wild type (WT) and
T8A-MiRP1 containing channels

| Channel | Activation | Activation | Deactivation | Deactivation | Deactivation ratio |
|---|---|---|---|---|---|
| (No. of cells) | V_{1/2} (mV) | slope (mV) | τ_{fast} (s) | τ_{slow} (s) | I_{fast}/(I_{fast} + I_{slow}) |
| WT (10) | | | | | |
| Control | −20 ± 5 | 9.5 ± 1.4 | 0.4 ± 0.1 | 1.5 ± 0.4 | 0.5 ± 0.1 |
| SMX | −20 ± 6 | 9.8 ± 2.2 | 0.3 ± 0.1 | 1.4 ± 0.3 | 0.5 ± 0.2 |
| T8A (10) | | | | | |
| Control | −25 ± 6 | 9.0 ± 1.2 | 0.6 ± 0.2 | 1.4 ± 0.4 | 0.5 ± 0.1 |
| SMX | −21 ± 5 | 9.4 ± 2.1 | 0.15 ± 0.04 | 0.9 ± 0.3 | 0.5 ± 0.1 |

Legend

Activation and deactivation kinetics were estimated in whole-cell recordings in the absence and presence of 633 μg/ml SMX Employing the protocol in FIG. 2, peak tail currents at −40 mV were normalized and fit to the Boltzmann function: 1/{1+exp[(V_{1/2}−V)/Vₛ]} where V_{1/2} is half maximal voltage and Vₛ the slope factor in mV. Deactivation kinetics were fit to a double exponential function (I₀+I_{f}e(−t/τ_{f})+Iₛe^{(-t/τ}s⁾) at −40 mV after a pre-pulse to 20 mV as in FIG. 3.

What is claimed:

1. An isolated polynucleotide sequence selected from the group consisting of:
(a) a nucleotide sequence that encodes an MiRP1 polypeptide that contains a mutation that causes long QT syndrome (LQTS), or biologically active fragments thereof that retain the mutation;
(b) a nucleotide sequence that encodes an MiRP1 polypeptide that contains a mutation at the amino acid corresponding to position 116 of the MiRP1 polypeptide described by SEQ ID NO: 4, or biologically active fragments thereof that retain the mutation;
(c) a nucleotide sequence that encodes an MiRP1 polypeptide that wherein the amino acid corresponding to position 116 of the MiRP1 polypeptide described by SEQ ID NO: 4 is valine or, or biologically active fragments thereof that retain the mutation;
(d) a nucleotide sequence that encodes an MiRP1 polypeptide described by SEQ ID NO: 2, or biologically active fragments thereof that retain the mutation; and
(e) a nucleotide sequence that is described by SEQ ID NO: 1, or fragments thereof that retain the mutation.

2. A vector comprising the polynucleotide of claim 1.

3. A cell transfected with the polynucleotide of claim 1.

4. A cell transfected with the vector of claim 2.

5. The cell of claim 4, wherein the cell is selected from the group consisting of prokaryotic and eukaryotic cells.

6. The eukaryotic cell of claim 5, wherein the cell is a Chinese Hamster Ovary (CHO) cell.

7. A method of producing a polypeptide comprising:
(a) culturing the cell of claim 3; and
(b) isolating said polypeptide.

8. An isolated polypeptide selected from the group consisting of:
(a) a polypeptide comprising the amino acid sequence described by SEQ ID NO: 4
wherein the amino acid at position 116 is not alanine;
(b) a polypeptide comprising the amino acid sequence described by SEQ ID NO:4
wherein the amino acid at position 116 is valine; and
(c) a polypeptide encoded by the polynucleotide of claim 1.

9. A nucleic acid probe that specifically hybridizes to the polynucleotide of claim 1 under stringent hybridization conditions wherein said hybridization conditions prevent said nucleic acid probe from hybridizing to the polynucleotide described by SEQ ID NO: 3.

10. The nucleic acid probe of claim 9 that consists of between 10-100 nucleotides.

11. The nucleic acid probe of claim 10 that comprises at least 10 contiguous nucleotides of SEQ ID NO: 1.

12. A nucleic acid probe that specifically hybridizes to the polynucleotide described by SEQ ID NO: 5 under stringent hybridization conditions wherein said hybridization conditions prevent said nucleic acid probe from hybridizing to the polynucleotide described by SEQ ID NO: 3.

13. The nucleic acid probe of claim 12 that consists of between 10-100 nucleotides.

14. The nucleic acid probe of claim 13 that comprises at least 10 contiguous nucleotides of SEQ ID NO: 5.

15. A nucleic acid probe that specifically hybridizes to the polynucleotide described by SEQ ID NO: 7 under stringent hybridization conditions wherein said hybridization conditions prevent said nucleic acid probe from hybridizing to the polynucleotide described by SEQ ID NO: 3.

16. The nucleic acid probe of claim 15 that consists of between 10-100 nucleotides.

17. The nucleic acid probe of claim 16 that comprises at least 10 contiguous nucleotides of SEQ ID NO: 7.

18. A nucleic acid probe that specifically hybridizes to the polynucleotide described by SEQ ID NO: 9 under stringent hybridization conditions wherein said hybridization conditions prevent said nucleic acid probe from hybridizing to the polynucleotide described by SEQ ID NO: 3.

19. The nucleic acid probe of claim 18 that consists of between 10-100 nucleotides.

20. The nucleic acid probe of claim 19 that comprises at least 10 contiguous nucleotides of SEQ ID NO: 9.

21. A method for diagnosing a polymorphism that causes drug-induced LQTS, comprising the step of hybridizing the nucleic acid probe of one of claims 9-20 to an isolated sample of a patient's DNA or RNA, under stringent hybridization conditions that allow hybridization of said probe to nucleic acid comprising said polymorphism but which prevent hybridization of said probe to the polynucleotide described by SEQ ID NO: 3, wherein the presence of a hybridization signal indicates the presence of said polymorphism.

22. The method according to claim 21 wherein the patient's DNA or RNA has been amplified and said amplified DNA or RNA is hybridized.

23. The method according to claim 22 wherein hybridization is performed in situ.

24. A method for diagnosing a polymorphism that causes drug-induced LQTS, comprising the DNA sequencing of a patient's KCNE2 gene wherein a mutation at a nucleotide position encoding amino acid positions 8, 54, 57, or 116 of the MiRP1 polypeptide, described by SEQ ID NO: 4, indicates the presence of said polymorphism.

25. A method for diagnosing a polyrmorphism that causes drug-induced LQTS, comprising the DNA sequencing of a patient's MiRP1 gene, wherein detection of mutations at amino acid positions 8, 54, 57, or 116 of said MiRP1 polypeptide, described by SEQ ID NO: 4, indicates the presence of said polymorphism.

26. An antibody that specifically binds to a mutant MiRP1 polypeptide but not to the polypeptide described by SEQ ID NO: 4.

27. The antibody of claim 26 that specifically binds to the polypeptide described by SEQ ID NO: 2.

28. The antibody of claim 26 that specifically binds to the polypeptide described by SEQ ID NO: 6.

29. The antibody of claim 26 that specifically binds to the polypeptide described by SEQ ID NO: 8.

30. The antibody of claim 26 that specifically binds to the polypeptide described by SEQ ID NO: 10.

31. The antibody of one of claims 26-30 wherein the antibody is a monoclonal antibody.

32. A method for diagnosing a polymorphism that causes drug-induced LQTS, comprising detecting the presence of a mutant MiRP1 polypeptide by contacting a sample containing a patient's MiRP1 polypeptide with the antibody of one of claims 26-30, wherein the presence of a positive reaction indicates said polymorphism.

33. A nonhuman, transgenic animal comprising the polynucleotide of claim 1.

34. A method of detecting compounds that are useful in treating or preventing LQTS, said method comprising:
(a) placing cells expressing wild-type HERG and wild-type KCNE2 into a bathing solution to measure current;
(b) measuring an induced K+ current in the cells of step (a);
(c) placing cells expressing wild-type HERG and mutant KCNE2 into a bathing solution to measure current;
(d) measuring the induced K+ current in cells of step (c);
(e) adding a candidate compound to the bathing solution of step (c);
(f) measuring an induced K+ current in the cells of step (e); and
(g) determining whether said candidate compound produces an induced K+ current more or less similar to the induced K+ current measured in cells expressing wild-type HERG and wild-type KCNE2 as compared to the current measured in cells expressing wild-type HERG and mutant KCNE2 in the absence of said candidate compound, wherein the candidate compound that produces a current more similar to the current observed in cells expressing wild-type HERG and wild-type KCNE2 is useful in treating or preventing LQTS.

35. The method of claim 36 wherein said mutant KCNE2 is selected from the group consisting of:
(a) a polynucleotide that encodes a mutant MiRP1 polypeptide described by SEQ ID NO: 2;
(b) a polynucleotide that encodes a mutant MiRP1 polypeptide described by SEQ ID NO: 6;
(c) a polynucleotide that encodes a mutant MiRP1 polypeptide described by SEQ ID NO: 8;
(d) a polynucleotide that encodes a mutant MiRP1 polypeptide described by SEQ ID NO: 10;
(e) a polynucleotide described by SEQ ID NO: 1;
(f) a polynucleotide described by SEQ ID NO: 5;
(g) a polynucleotide described by SEQ ID NO: 7; and
(h) a polynucleotide described by SEQ ID NO: 9.
